(19) United States
(12) Patent Application Publication
(10) Pub. No.: US 2015/0191408 A1
(43) Pub. Date: Jul. 9, 2015

(54) TRIPTYCENE MONOMER AND TRIPTYCENE CONTAINING POLYESTERS AND POLYURETHANES

(71) Applicants: S. Richard Turner, Blacksburg, VA (US); Yanchun Liu, Blacksburg, VA (US); Zhengmian Chang, Blacksburg, VA (US)

(72) Inventors: S. Richard Turner, Blacksburg, VA (US); Yanchun Liu, Blacksburg, VA (US); Zhengmian Chang, Blacksburg, VA (US)

(21) Appl. No.: 13/588,282

(22) Filed: Aug. 17, 2012

Related U.S. Application Data
(60) Provisional application No. 61/525,223, filed on Aug. 19, 2011.

(51) Int. Cl. C07C 43/23; C08G 18/32; C08G 63/78; C08G 18/72; C08G 63/00; C08G 69/44

(52) U.S. Cl. CPC C07C 43/23; C08G 63/00; C08G 69/44; C08G 63/78; C08G 18/72; C08G 18/3215; C07C 2103/90

(57) ABSTRACT

The primary diol triptycene derivative triptycene-1,4-hydroquinone-bis(2-hydroxyethyl) ether (TD) is provided, as are methods of using the same to synthesize polyesters and polyurethanes, and polyesters and polyurethanes synthesized therewith.

### CROSS-REFERENCE TO RELATED APPLICATIONS

This application claims benefit of U.S. provisional patent application 61/525,223, filed Aug. 19, 2011, the complete contents of which is hereby incorporated by reference.

### STATEMENT OF FEDERALLY SPONSORED RESEARCH AND DEVELOPMENT

This invention was made, in part, with government support under Grant No W911NF-062-2-0014 awarded by the United States Army Research Laboratory. The government has certain rights in the invention.

### BACKGROUND OF THE INVENTION

1. Field of the Invention

The invention generally relates to triptycene-1,4-hydroquinone-bis(2-hydroxyethyl) ether (TD), a primary diol triptycene derivative. In particular, the invention provides TD; methods of synthesizing polyesters, polyurethanes and polyesteramides using TD; and polyesters, polyurethanes and polyesteramides synthesized using TD and/or containing TD.

2. Background of the Invention

Material scientists have been pursuing the enhancement of mechanical properties to produce high performance polymers for a wide range of applications. Incorporation of rigid structures into a polymer backbone to enhance properties has been studied extensively by many researchers.¹⁻³ Polymers containing such rigid building blocks usually show not only increased Tg but also decreased ductility. For example, adamantyl building blocks usually raise the Tg, but also lower the ductility of a polymer by reducing the flexibility of the polymer backbone and intermolecular chain entanglements.⁴⁻⁷ A variety of bisphenol derivatives are also well known to produce high Tg polymers.⁸⁻¹⁰ However, a recent report from Swager, Thomas, et al. shows that incorporation of triptycene, a rigid aromatic cyclic structure, gives an increase in both modulus and ductility even at a low temperature of −30° C. when incorporated into certain polyester backbones.^{11,12} The authors hypothesized that neighboring chains can lie in a V-shaped cleft of the triptycene units and that this provides a mechanism for molecular interlocking and is the origin of these normally divergent mechanical properties.¹¹ Unlike more general intermolecular interactions such as hydrogen bonding and ionic clustering, this “mechanical interlocking” effect has been claimed in only a few polymers where triptycene units are incorporated by a solution polymerization process of the triptycene 1,4-hydroquinone.

Previous work on triptycene containing polyesters in the late 1960's from DuPont and Eastman Kodak resulted in polymers with significantly enhanced glass transition temperatures and increased brittleness as evidenced by the reported brittle nature of cast films.^{13,14} Both of these early reports were based on incorporation of a triptycene monomer with a 9,10 functionality for polymerization into the various polymer backbones studied. In contrast the Swager and Thomas work is based on the use of 1,4-hydroquinone triptycene structure which significantly changes the monomer structure and polymer backbone structure. Also in the recent work a long aliphatic spacer, incorporated from a comonomers, was found to be necessary to bring this proposed mechanical interlocking mechanism into operation. The combination of decanediol and the 1,4-hydroquinone triptycene units in the polyester chain led to these unusual properties.

1,4-Hydroquinone triptycene has also been incorporated into polycarbonates (a) and polysulfones (b).
(a) Tsui, N.; Yang, Y.; Mulliken, A.; Torun, L.; Boyce, M.; Swager, T.; Thomas, E. Polymer 49(2008) 4703-4712.
(b) Ritai, S.; Breen, C.; Solis, D.; Swager, T. Chem Mater. (2006) 18, 21-25, the complete contents of which are herein incorporated by reference.

There remains a need in the art for development of high performance polymers with enhanced mechanical properties for a wide range of applications especially polymers with both high modulus values and good ductility.

### SUMMARY OF THE INVENTION

The present invention provides a new primary diol triptycene derivative: triptycene-1,4-hydroquinone-bis(2-hydroxyethyl) ether (TD). The invention also provides methods of using TD to synthesize polyesters and polyurethanes, and polyesters and polyurethanes synthesized using TD and/or which contain TD.

In one embodiment, the use of TD advantageously permits the facile melt phase preparation of copolyesters with excellent mechanical properties. TD polyesters exhibit enhanced elongation and modulus parameters, and thus products formed from these copolymers (even thin sheets) exhibit increased toughness and resistance to impact failure and deformation. TD polyesters also exhibit higher Tg and heat deflection temperatures than do comparable prior art polymers, making them useful for the manufacture of products for which resistance to thermal deformation is required or advantageous.

It is an object of this invention to provide a new reactive triptycene monomer (TD) with primary alcohol end groups which enables the preparation of new and useful polyesters polyesteramides, and polyurethanes with useful balances on stiffness and ductility that are not available from existing triptycene monomers such as 1,4-hydroquinone triptycene.

### BRIEF DESCRIPTION OF THE DRAWINGS

FIG. 1. ¹H nuclear magnetic resonance (NMR) spectra of poly[100(DMCD)(74)(EG)26(TD)].

FIG. 2. SEC trace of poly[100(DMCD)(74)(EG)26(TD)]

FIGS. 3A and B. Tensile properties of A, poly[100(DMCD)74(EG)26(TD)]; and B, poly[100(DMCD)74(EG)26(HBE)] (right) at 23° C.

FIGS. 4 A and B. Ambient temperature tensile properties of A, poly[100(DMCD)75(BD)25(TD)]; and B, poly[100(DMCD)75(BD)25(BHPT)].

FIG. 5. X-ray diffraction traces of poly (100(DMCD)74(BD)26(TD) films before and after stretching.

FIGS. 6A and B. DMA of poly[100(DMCD)75(BD)25(TD/BHPT/BHPC/BHPS/HBE)]. A, GPa; B, tan delta.

### DETAILED DESCRIPTION

Triptycene-1,4-hydroquinone-bis(2-hydroxyethyl) ether (TD), a new triptycene diol, has been synthesized. This TD is useful for the synthesis of polymers such as polyesters and polyurethanes.

### Polyesters

In one embodiment, the TD is used to prepare copolymeric polyesters, which may be referred to herein as “TD copolyesters” or “TD polyesters” or simply “copolyesters” or “polyesters”. By “polyester” we mean polymers that contain an ester functional group in their main chain, Polyesters are produced chiefly by the reaction of dibasic acids with dihydric alcohols, and the exemplary polyesters of the invention may be synthesized using two dihydric alcohols, one of which is the new TD described herein or use of the TD as the sole dihydric alcohol. The methods for producing TD copolyesters according to the invention generally comprise conducting an esterification reaction or a transesterification reaction between a dicarboxylic acid or an ester-forming derivative thereof (e.g. a diester) and the TD glycol or with at least two glycols, at least one of which is the TD of the invention.

Exemplary dicarboxylic acids which may be used include but are not limited to: aliphatic dicarboxylic acids exemplified by succinic acid, glutaric acid, adipic acid and dodecanedicarboxylic acid and their ester-forming derivatives such as dialkyl esters; and aromatic dicarboxylic acids exemplified by terephthalic acid, isophthalic acid, naphthalene dicarboxylic acid, dimethyl-2,6-naphthalenedicarboxylate (NDC), dimethyl-2,7-naphthalenedicarboxylate, dimethyl-2,3-naphthalenedicarboxylate, dimethyl 1,4-cyclohexanedicarboxylate, and their ester-forming derivatives such as dialkyl esters; 4,4′-biphenyldicarboxlic acid and dimethyl-4,4′-biphenyldicarboxylate, 1,3-cyclohexane dicarboxlic acid, as well as combinations thereof.

Exemplary glycols that may be used in combination with TD for copolyester synthesis include but are not limited to: ethylene glycol (EG); 1,4-butanediol (BD); 1,6-hexanediol (HD); 2,2-bis(4-hydroxyphenyl)propane also known as bisphenol A; 1,1-bis(4-hydroxyphenyl)cyclohexane also known as bisphenol Z; 4,4-biphenol. hydroquinone bis(2-hydroxyethyl) ether (HBE); bis[4-(2-hydroxyethoxyl)phenyl]sulfone (BHPS); bis[4-(2-hydroxyethoxy)-phenyl]4,4′-cyclohexylidene (BHPC); 1,1-bis(2-hydroxyethoxy)phenyl-3,3,5-trimethylcyclohexane (BHPT); propylene glycol, diethylene glycol, triethylene glycol, butylene glycol, 1,4-cyclohexanedimethanol, neopentyl glycol, tetramethyl cyclobutane diol, etc

TD copolyester forming reactions are generally carried out in the presence of a catalyst, examples of which include but are not limited to: various titanium (Ti) based compounds including tetra-n-propyl titanate, tetraisopropyl titanate, tetra-n-butyl titanate, tetraphenyl titanate, tetracyclohexyl titanate, tetrabenzyl titanate, tetra-n-butyl titanate tetramer, titanium acetate, titanium glycolates, titanium oxalates, sodium or potassium titanates, titanium halides, titanate hexafluorides of potassium, manganese and ammonium, titanium acetylacetate, titanium alkoxides, titanate phosphites, titanium n-butoxide as described in published US patent application 20120172571 (Tabata), the complete contents of which is herein incorporated by reference; nano-layered titanate in exfoliated form as described in US patent application 20120010382 (Bashir), the complete contents of which is herein incorporated by reference; catalysts based on antimony (Sb) such as antimony triacetate or antimony trioxide; germanium dioxide, etc.

The co-polyesters of the invention may be synthesized by any method or combination of methods known in the art. Exemplary methods include but are not limited to melt polycondensation, solid phase polycondensation (if the polyester is semicrystalline, conducting steps under increased or reduced pressure or under an inert gas atmosphere, etc. For example, the reactants may be esterified or transesterified and then subjected to melt polycondensation, The polyester, if semi-crystalline, obtained by these processes may be subjected to solid phase polymerization. Of these processes, a preparation process using melt polycondensation preferable is because the various components can be copolymerized in the absence of solvents which can be toxic or need to be recycled or disposed of. As one example of the process for preparing copolyesters, a process comprising performing esterification or transesterification to prepare a low molecular weight polymer, subjecting the low polymer to melt polycondensation and then further subjecting the polycondensate to solid phase polymerization, if the polyester is semicrystalline, to increase its molecular weight, may be used.

For esterification, dicarboxylic acids and diols may be directly esterified at the same time or successively at a temperature of preferably e.g. about 130 to 220° C. under pressure or at atmospheric pressure. The amounts or ratios of reactants used in such reaction are generally chosen with the more volatile diol in excess so the polycondensation can be driven to high conversion and high molecular weight with heat and under vacuum. The esterification reaction may be carried out without adding any catalyst or may be carried out in the presence of a catalyst, for example, an acid, such as concentrated sulfuric acid or p-toluenesulfonic acid, or a metal complex. Transesterifications (e.g. using one or more diesters, which may be the same or different) are typically carried out at a temperature of from about 130 to 220° C. at atmospheric pressure, usually in the presence of various metal complexes, such as manganese acetate and zinc acetate. Then, the lower molecular weight polymer obtained by the above processes is subjected to melt polycondensation in the presence of e.g. a polymerization catalyst and a stabilizer in the temperature range of 150 to 300° C., preferably 190 to 280° C., under reduced pressure conditions of not more than about 10 Torr, preferably not more than about 2 Torr, for a period of 1 hour to 24 hours, preferably 2 hours to 12 hours, with stirring.

Descriptions of methods for synthesizing copolyesters are known to those of skill in the art, for example, in U.S. Pat. No. 6,120,889 (Turner, et al.), U.S. Pat. No. 4,093,603 (Jackson, et al.), U.S. Pat. No. 7,026,027 (Turner, et al.), U.S. Pat. No. 5,681,918 (Adams, et al.), the complete contents of each of which are hereby incorporated by reference.

In some embodiments, the polyesters are synthesized by melt polycondensation. The primary alcohol end groups of the TD advantageously enables preparation of polyesters in the melt phase without having to use expensive, toxic solvents, ester activating groups like corrosive acid chlorides, etc. This technique is well-known to those of skill in the art, e.g. see Fradet, A.; Tessier, M. Chapter 2 “Polyesters” in “Synthetic Methods in Step-Growth Polymerization” Editors Rogers, M.; Long, T.; Wiley Interscience 2003. Specific examples of copolyesters that may be prepared as described herein include but are not limited to copolyesters based on TD with 1,4-cyclohexane dicarboxlic acid and other diols such as 1,4-CHDM, 1,4-butanediol, 1,6-hexanediol. Copolyesters based on mixtures of adipic acid, 1,4-cyclohexane dicarboxylic acid with TD and other diols as described above.

### Polyesteramides

In some embodiments of the invention, the TD copolyesters of the invention are TD co-polyesteramides made with and which incorporate the TDs described herein. By “polyesteramide” we mean a polymer containing both ester and amide groups. The co-polyesteramides of the invention are generally formed as described above for co-polyesters, except that synthesis is carried out using at least one amide-generating group in the reaction mixture. Generic methods of synthesizing polyesteramides are known in the art. For example, the synthesis of polyesteramides is described in U.S. Pat. No. 5,672,676 (Turner), the complete contents of which are herein incorporated by reference. Suitable amide-generating starting materials include but are not limited to diamines which are the amine-equivalents of the aforementioned dicarboxylic acids; diamines such as ethylenediamine, propylenediamine, tetramethylenediamine, pentamethylenediamine, hexamethylenediamine, 1,4-cyclohexane-bismethylamine, bis(p-amino-cyclohexyl)methane as well as dimer fatty diamines (derived from dimer fatty acids); various aromatic diamines including materials derived from benzene, toluene and other substituted aromatic materials, e.g. 2,6-tolylenediamine, 4,4-diphenylmethanediamine and xylylenediamine; diamines which contain one or two secondary amino groups; heterocyclic diamines, for example piperazine; branched diamines, such as 3-methyl pentane diamine; etc.

### Polyurethanes

The primary OH groups on the triptycene monomer diol (TD) renders it a viable chain extender for the manufacture of polyurethanes. As is known in the art, the choice of chain extender helps to determine flexural, heat, and chemical resistance properties of polyurethanes. “Polyurethane” (PUR and PU) as used herein refers to a polymer composed of a chain of organic units joined by urethane links. These linear polymeric chains contain low polarity segments (called soft segments), alternating with (usually) shorter, high polarity segments (called hard segments). Both types of segments are linked together by covalent links, so that they actually form block-copolymers.

Polyurethanes are typically formed by the polymerization of one or more polyisocyanates, typically diisocyanates, and one or more polyols. As used herein, “polyisocyanate” refers to a molecule with two or more isocyanate functional groups (R—(N═C═O)n, where n≧2) and “polyol” refers to a molecule with two or more hydroxyl functional groups (R′—(OH)n, n≧2). These reactions also require “chain extenders”. Chain extenders are generally multi-functional molecules that are included as additives in the reaction during one or more of the described processing steps with the purpose of further coupling polycondensates chains or “re-coupling” polycondensate chains that have depolymerized to some degree. In some embodiments, the TD of the invention is used as a chain extender during polyurethane formation.

In some embodiments, the polyol used for producing polyurethane comprise one or more TDs in the polyol backbone. The “backbone” of the polyol refers to the continuously linked portion of the polymer, which typically contains many carbon atoms and usually oxygen atoms.

Many polyisocyanates which can be used in the practice of the invention are known in the art. These include various aromatic, aliphatic and cycloaliphatic polyisocyanates, examples of which include but are not limited to: toluene diisocyanate (TDI) and isomers thereof (e.g. 2,4- and 2,6-diisocyanatotoluene isomers); diphenylmethane diisocyanate (MDI) an isomers thereof (e.g. 4,4′-MDI, 2,4′-MDI, and 2,2′-MDI); p-phenylene diisocyanate (PPDI), naphthalene diisocyanate (NDI), o-tolidine diisocyanate (TODI); 1,6-hexamethylene diisocyanate (HDI); 1-isocyanato-3-isocyanatomethyl-3,5,5-trimethyl-cyclohexane (isophorone diisocyanate, IPDI); 4,4′-diisocyanato dicyclohexylmethane (H12MDI or hydrogenated MDI); cyclohexane diisocyanate (CHDI), tetramethylxylene diisocyanate (TMXDI); 1,3-bis(isocyanatomethyl)cyclohexane (H6XDI); etc. as well as prepolymers, biurets, dimers, trimers, adducts, isomers and mixtures thereof.

Many polyols are known which can be used in the production of polyurethanes, including but not limited to: “soft diols” such as polyethylene glycol (PEG), polytetramethylene glycol (PTMG), polypropylene glycol (PPG); various polyester polyols and polyethers polyols and blends thereof; polycarbonate polyols, polycaprolactone polyols, polybutadiene polyols, polysulfide polyols; various natural oil polyols (e.g. derived from castor oil and other vegetable oils); and various fluorinated (FEVE) polyols; etc., as well as mixtures thereof.

The reaction mixture may further comprise various initiators, catalysts, surfactants, blowing agents such as CO₂, defoaming agents (for example, solutions of polysiloxane defoamers such as, for example, the products sold by BYK Chemie under the names BYK A506 and BYK A530); cross linkers, flame retardants, light stabilizers, fillers, pigments, clays, catalysts and other additives that are known to those of skill in the art. Exemplary catalysts that may be employed include but are not limited to tertiary amines, such as dimethylcyclohexylamine; trialkylamines (tetramethylbutanediamine, bis(2-dimethylaminoethyl)-ether, etc.); aliphatic polyamines; Mannich bases; diazabicycloundecene (DBU); tin salts (tin octoate, dibutyltin laurate, etc.); mercury salts; zinc salts; lead salts; calcium salts; magnesium salts; N-alkylmorpholines; phosphines; carboxylates (magnesium carboxylate, potassium carboxylate, aluminum carboxylate, etc.); various organometallic compounds such as dibutyltin dilaurate or bismuth octanoate; 1,4-diazabicyclo[2.2.2]octane (diazabicyclooctane, also called DABCO or TEDA); bis-(2-dimethylaminoethyl)ether; potassium octoate; etc.

Additional chain extenders may be used in addition to TD (i.e. with or in combination with) TD, including but not limited to: ethylene glycol; 1,4-butanediol (1,4-BDO or BDO); 1,6-hexanediol; 1,4-cyclohexane dimethanol; hydroquinone bis(2-hydroxyethyl) ether (HQEE); etc. Chain extenders may also be diamines which form urea linkages and the final polymer is a poly(urethane urea), see U.S. Pat. No. 8,080,626. To obtain polyurethane elastomer materials, several different processes are possible: e.g. a “one shot” process is conceivable, during which all the components mentioned above are added in a single step; preferably, 2-step reaction involving formation of a prepolymer is employed. During the first step, the long diol or mixture of long dials is reacted with an excess of a diisocyanate in order to obtain a prepolymer possessing isocyanate functional groups. The prepolymer is then reacted with an amount of chain extender (TD, or TD in combination with one or more other chain extenders), the amount of TD (with or without additional chain extenders) being sufficient to attain polymers with the desired characteristics (elasticity, adhesiveness, etc.). In some processes of the invention, the polyol or polyetheramine, chain extender composition, and when used, optional ingredients, are blended together to form a first mixture, followed by blending this first mixture with the isocyanate to form a second mixture; and then the second mixture is allowed to cure. In other processes of this invention, the isocyanate and the polyol or polyetheramine are blended together to form a prepolymer, which prepolymer is then mixed together with the chain extender composition to form the desired polymer. In still other processes of the invention, the isocyanate is mixed with polyol or polyetheramine to form a quasiprepolymer; polyol or polyetheramine is mixed with the chain extender composition to form a mixture; and then the mixture is mixed with the quasiprepolymer to form the desired polymer. Thus, the chain extender composition is reacted with an aromatic polyisocyanate and at least one polyol and/or at least one polyetheramine or with a prepolymer or a quasiprepolymer of the isocyanate and the polyol or polyetheramine. Other methods for synthesizing polyurethanes are described, for example, in U.S. Pat. No. 8,080,626 (Wiggins, et al.) and U.S. Pat. No. 6,780,958 (DeGuia), the complete contents of each of which are herein incorporated by reference.

### Properties and Uses of the Polymers of the Invention

The polyesters of the invention display several properties which are advantageous for the production of materials therefrom. For example, the polyesters exhibit enhanced elongation and modulus properties, allowing the production of thinner sheets of polyester that still are resistant to deformation, and hence maintain their shape under pressure. Such transparent, amorphous copolyesters could be used for window glazing, point of purchase displays, optical sheets, etc. In other words, the enhanced elongations signal enhanced toughness or resistance to impact failure. Thus, polyesters containing TD may be used as matrix polymers in composites in order to enhance impact resistance of the composite material. The inclusion of TD in polyesters results in higher T_{g} values and hence increased heat deflection temperatures. TD polyesters are thus suitable for use in materials requiring resistance to thermal deformations. For example, the polyesters could be used in combination with ultra high molecular weight polyethylene fibers or Kevlar fibers to enhance impact resistance of there materials, leading to the production of light-weight composites for use in body armor (e.g. combat armor, soft-body armor) and other armored applications, e.g. for vehicle manufacture, helmets, sports gear, etc. The polyesters of the invention may be used in any of the many known polyester products, including but not limited to various textiles (clothing, sheets, outerwear, etc.), and other consumer, commercial or health-related goods (sails, synthetic tissue, ropes, carpets and rugs, to name a few).

The polyurethanes of the invention have similar applications e.g. in the manufacture of thermoplastic elastomers, adhesives, and sealants. In particular, these polyurethanes may be used as fiber adhesives for ultra-high-molecular-weight polyethylene (UHMWPE) fiber composites and Kevlar fiber systems when strong adhesion with energy absorbing properties is required. Products that may be fabricated using the TD polyurethanes of the invention include but are not limited to: various flexible and rigid foams; elastomeric wheels and tires; automotive parts such as suspension bushings; electrical potting compounds; high performance adhesives, surface coatings and sealants; synthetic fibers; carpets and carpet underlay; hard-plastic parts e.g. for electronic instruments, protective sheeting, tarps; various opaque and transparent molded “plastics”; polyurethane fiber based composites, etc.; and for a plethora of other applications that will occur to those skilled in the art.

### EXAMPLES

### Example 1

### Melt-Phase Synthesis and Properties of Triptycene Containing Copolyesters

### 1. Overview

The experiments described herein explore methods to raise the Tg values of aliphatic polyesters based on 1,4-cyclohexane dicarboxylic acid (via 1,4-DMCD) without negatively impacting the mechanical properties of these materials. The number of methylene groups in the aliphatic co-diol was varied from two to six (ethylene diol to hexane diol). Other bulky hydroxyethoxylated bisphenol derivatives were incorporated into identical polyester backbones and these polyesters were prepared as comparative examples to the TD containing polyesters.

### 2. Materials and Measurements

Anthracene (97%) was purchased from Aldrich and recrystallized from xylene. Ethylene glycol (≧99%), 1,4-butanediol (99%), 1,6-hexanediol (99%), p-benzoquinone, hydroquinone bis(2-hydroxyethyl) ether (98%) were purchased from Aldrich and used as received. Dimethyl 1,4-cyclohexanedicarboxylate (DMCD) (cis/trans=3/1) was donated by Eastman Chemical Company.

All measurements were performed in Virginia Tech (Blacksburg, Va.) except for the elemental analysis, which was done by Atlantic Microlab, Inc. (Norcross, Ga.). NMR spectra were determined at 25° C. at 400 MHz with an INOVA spectrometer. Molecular weights of the synthesized polymers were determined using size exclusion chromatography (SEC) with a refractive index (RI) detector and viscometer DP detector. SEC measurements were performed at 30° C. in chloroform with a sample concentration 5.0 mg/ml at a flow rate of 1.0 ml/min. Thermogravimetric analysis (TGA) was conducted under nitrogen from 25 to 600° C. at a heating rate of 10° C./min using a TGA Q500 of TA Instruments. Differential scanning calorimetry (DSC) was conducted using a DSC Q2000 of TA instruments. DSC data were obtained from −20° C. to 300° C. at heating/cooling rates of 20° C./min under nitrogen circulation. The glass transition temperature was determined from analysis of the second heating cycle. Dynamic mechanical analysis (DMA) of samples was conducted using a DMA Q800 of TA instruments at a heating rate of 5° C./min from −150° C. to 100° C. while they were deformed (10 micrometer amplitude) in the tension mode at a frequency of 1 Hz under nitrogen. Tensile measurements at room temperature were performed on an Instron Model 4400 Universal Testing System equipped with a 1KN load cell. Tensile measurements at low temperatures (0° C. or −25° C.) were performed on an Instron 5800R and Thermotron Testing System equipped with a load capacity of 1KN. The film samples were prepared using a PHI Model GS 214-C-7 compression molding press at 70° C. above T_{g} for 15 min. After the film samples were cooled down in ambient air, they were stored in a desiccator at ambient temperature. The molecular weights of the prepared film samples were unchanged from the original samples before compression molding. The film samples were dried in vacuum 24 h and then were cut to a dog bone shape at 40×4×0.3 mm (length×width×thickness) for tensile tests. The samples were tested at a rate of 15 mm/min using an initial grip-to-grip separation of 15 mm. Young's modulus was calculated from the linear part of the initial slope. All reported tensile data were averaged from at least three independent measurements and a standard deviation was also reported. An x-ray diffractometer was used to determine if any crystallinity existed within the cast films. For this experiment, the original films or stretched films were fixed on the platform and the x-ray diffraction was observed from the surface of films by use of the reflection mode.

### Preparation of Monomers

### 2.2.1 Synthesis of triptycene-1,4-quinone (1)

Triptycene 1,4-quinone (1) was prepared by following published literature procedures.^{15,16} The product was recrystallized from xylene and washed with cold xylene, then dried in a vacuum oven at 60° C. overnight. Finally pale yellow solid (1) was obtained with a yield of 88%, mp 230-232° C. (lit., 227-232° C.¹⁷), ¹H NMR (400 MHz; CDCl₃) δ ppm: 3.14 (s, 2H, COCH), 4.87 (s, 2H, Ar—CH), 6.31 (s, 2H, C═CH), 7.07-7.10 (m, 2H, Ar—H), 7.18-7.20 (m, 4H, Ar—H), and 7.35-7.41 (m, 2H, Ar—H).

### 2.2.2 Synthesis of triptycene-1,4-hydroquinone (2)

Triptycene-1,4-hydroquinone was obtained by the rearrangement of triptycene-1,4-quinone (1) in the solvent acetic acid at the boiling point in the presence of hydrobromic acid.¹⁵ Yield 90%, mp 340-342° C. (lit., 338-340° C.¹⁵). ¹H NMR (400 MHz; DMSO-d₆) δ ppm: 5.80 (s, 2H, Ar—CH), 6.31 (s, 2H, Ar—H), 6.96-6.98 (m, 4H, Ar—H), 7.38-7.40 (m, 4H, Ar—H), and 8.83 (s, 2H, Ar—OH).

### 2.2.3 Synthesis of triptycene-1,4-hydroquinone-bis(2-hydroxyethyl) ether (3)

Synthesis of triptycene-1,4-hydroquinone-bis(2-hydroxyethyl) ether (3) is shown in Scheme 1. A 500 ml two-necked flask charged with 30 g (0.105 mol) triptycene-1,4-hydroquinone (2), 1.45 g K₂CO₃ (0.0105 mol) in 250 ml N,N-dimethylformamide (DMF) was heated to reflux temperature (165° C.) under Argon, 18.46 g (0.210 mol) ethylene carbonate in 100 ml DMF was added into the flask drop by drop (about 1 hour). After that, the reaction mixture was stirred at reflux temperature for another 2 hours and then cooled to room temperature. A fine precipitate was obtained by pouring the reaction mixture into 800 ml of deionized water. The solid was filtered and washed completely with deionized water. A fine white solid (3) was obtained after recrystallization from methanol and drying under vacuum oven overnight. Yield: 89%, mp 238-239.3° C. NMR (400 MHz; DMSO-d₆) δ ppm: 3.74-3.77 (m, 4H, CH₂OH), 3.92-3.96 (m, 4H, ArO—CH₂), 4.94-4.97 (t, 2H, O—H), 5.95 (s, 2H, Ar—CH), 6.64 (s, 2H, Ar—H), 6.97-6.70 (m, 4H, Ar—H), 7.15-7.17 (d, 4H, Ar—H). ¹³C NMR (DMSO-d₆, 100 MHz) δ ppm: 46.81, 60.21, 72.11, 112.09, 124.13, 125.25, 135.71, 145.93, 148.64. Elemental analysis calculated: C, 76.99; H, 5.92. Found: C, 77.03; H, 5.89.

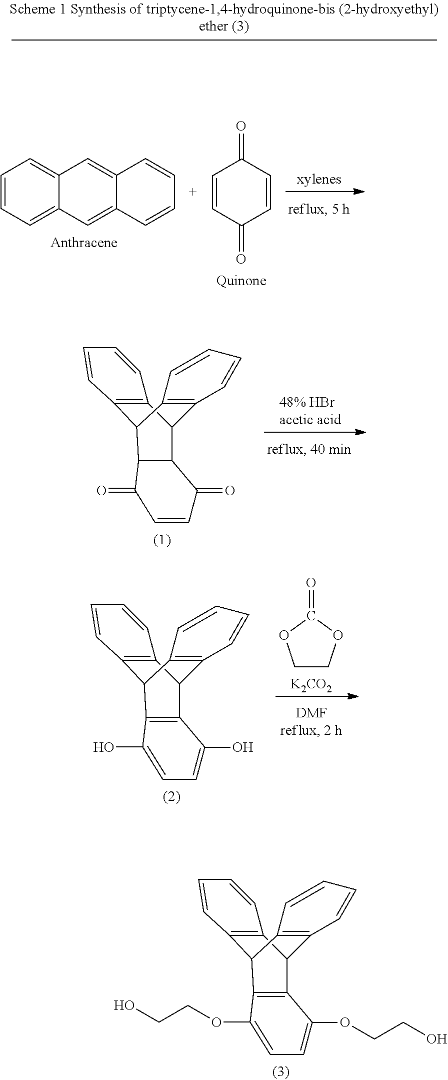

### 2.2.4 Synthesis of bis[4-(2-hydroxyethoxy)-phenyl]4,4′-cyclohexylidene (BHPC) (4)

Synthetic procedures of bis[4-(2-hydroxyethoxy)-phenyl]4,4′-cyclohexylidene (BHPC) (4) were similar to those of triptycene-1,4-hydroquinone-bis(2-hydroxyethyl) ether (3). Yield: 90%, mp 101-102° C. NMR (400 MHz; DMSO-d₆) δ ppm: 1.47-1.54 (m, 6H, CH₂CH₂CH₂CH₂CH₂), 2.07-2.10 (t, 2H, O—H), 2.20-2.22 (m, 4H, CH₂CCH₂), 3.90-3.94 (m, 4H, CH₂OH), 4.03-4.05 (m, 4H, ArO—CH₂), 6.80-6.83 (m, 4H, Ar—H), 7.15-7.17 (m, 4H, Ar—H).

### 2.2.5 Synthesis of 1, I-bis(2-hydroxyethoxy)phenyl-3,3,5-trimethylcyclohexane (BHPT) (5)

1,1-bis(2-hydroxyethoxy)phenyl-3,3,5-trimethylcyclohexane (BHPT) (5) was synthesized according to the literature procedure.⁹ The final product is a white glassy solid with a yield of 97%. No melting endothermic peak is observed in the DSC curves, the T_{g} of this monomer is 40° C. ¹H NMR (CDCl₃, 400 MHz) δ_{H} 7.23 (d, 2H, J=4.4 Hz), 7.10 (d, 2H, J=4.4 Hz), 6.80 (d, 2H, J=4.4 Hz), 6.75 (d, 2H, J=4.4 Hz), 3.88-4.05 (m, 8H), 2.63 (d, 1H, J=6.8 Hz), 2.39 (d, 1H, J=6.8 Hz), 2.10 (t, 1H, J=6.4 Hz), 1H, J=6.4 Hz), 1.95-2.02 (m, 1H), 1.91 (d, 1H, J=6.4), 1.37 (d, 1H, J=5.6 Hz), 1.13 (t, 1H, J=12.8 Hz), 0.96 (d, 6H, J=4.0 Hz), 0.86 (t, 1H, J=12.8 Hz), 0.36 (s, 3H)

### 2.3 Synthesis of Copolyesters

### 2.3.1 Preparation of Catalyst Solution

The titanium catalyst solution was obtained by mixing titanium n-butoxide with n-butanol in a dry bottle under nitrogen at a concentration of 0.06 g/ml based on Ti.

### 2.3.2 Abbreviation of Polyesters

The polymer nomenclature used herein is based on a polyester containing 100 mol % of diester and 100 mol % of diol. For example, the polymer designated by poly[100(DMCD)75(EG)25(TD)] means that this targeted polymer contains 100 mol % DMCD as the diester units and 75 mol % EG and 25 mol % TD as the diol units. The letters, stand for various monomers' abbreviation and the numbers indicate targeted mol % of monomers, respectively.

### 2.3.3 Polymerization

The triptycene dial (TD) (3) and the comonomer EG were copolymerized with DMCD by melt polycondensation reaction. The detailed procedure in Scheme 2, which is similar to a published method,⁹ is as follows (for poly[100(DMCD)75(EG)25(TD)]): 10 g (0.05 mol) of DMCD consisting of a trans/cis (1/3 molar ratio) mixture, 4.66 g (0.075 mol) of EG (100% excess) and 4.68 g (0.0125 mol) TD were charged to a two-necked 50 ml reaction vessel equipped with a mechanical stirrer, nitrogen inlet, and condenser. The reactor was placed in a Belmont metal bath with a temperature controller. Titanium n-butoxide catalyst (100 ppm with respect to the targeted polyester) was added via a syringe under nitrogen. A multi-step temperature procedure was used for the reaction, i.e. the reaction mixture was heated and stirred at 190° C. for 2 hours, 220° C. for 2 hours and 275° C. for 0.5 hour. Methanol was collected in a receiving flask. At the end, high vacuum (0.1-0.2 mm Hg) was applied to drive the reaction to high conversion for an additional 2 hours. Then the vacuum was discontinued and nitrogen was passed through the system. The polymer was allowed to cool to room temperature and was removed from the reaction flask. The polymer was dissolved in chloroform, and precipitated into methanol. The solid precipitate was obtained by vacuum filtration and was dried under vacuum at 30-60° C. overnight before characterization. The same procedure was employed to prepare poly[100(DMCD)(100−x)(EG)×(TD)].

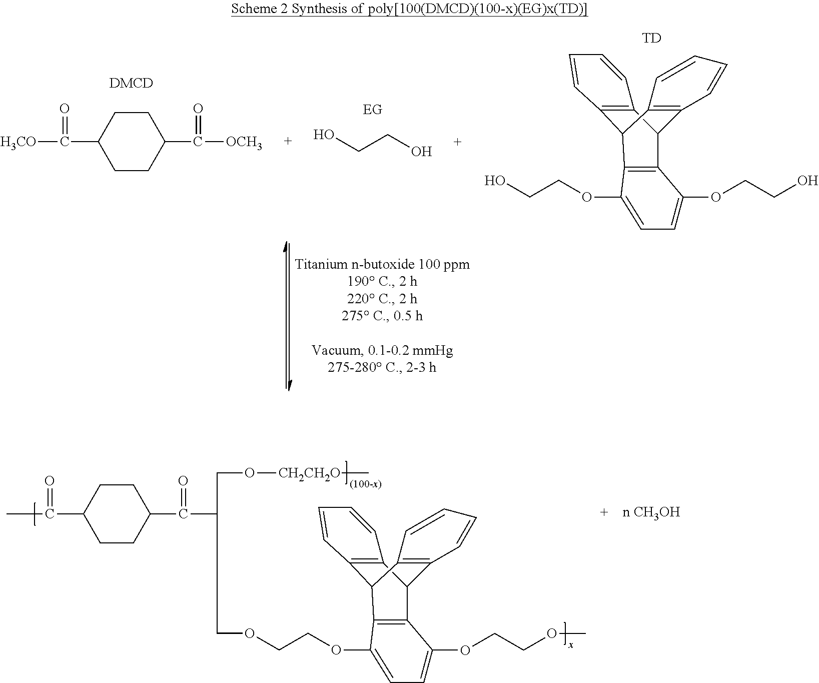

2.3.4 Synthesis of Poly[100(DMCD)(100−x)(EG)×(HBE)] for Comparative Purposes

Non-triptycene analogs also were synthesized for comparison. 1,4-bis(2-hydroxyethoxy)benzene (HBE) was used as a corresponding diol shown in Scheme 3. The experimental procedures are the same as described above.

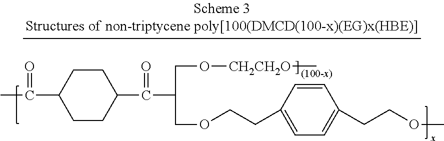

### 2.3.5 Synthesis of Poly[100(DMCD)75(BD or HD)25(TD)]

Copolyesters were prepared by the above mentioned procedure except that the content of TD and straight-chain alkanediols were fixed (see Scheme 4). Ethanediol was replaced by butanediol or hexanediol.

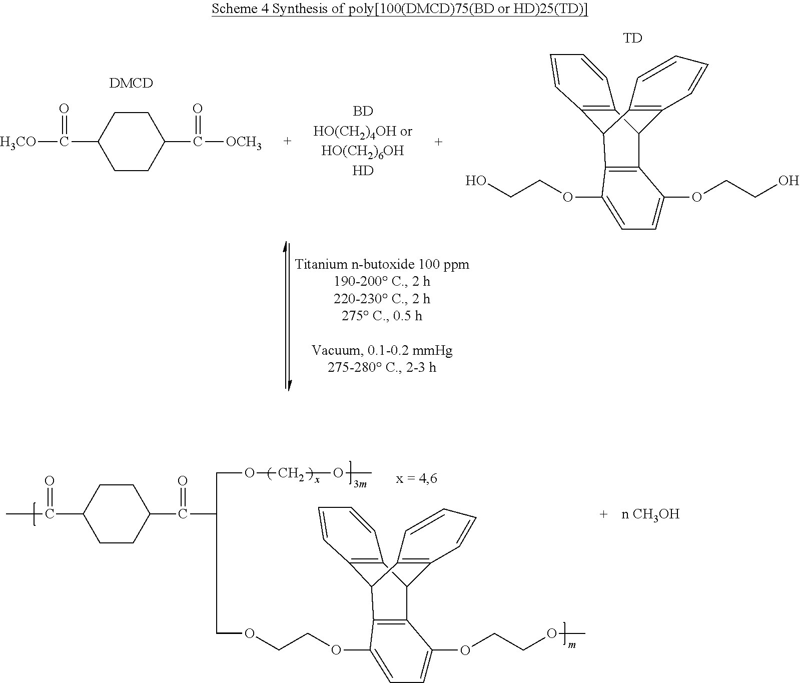

### 2.3.6 Synthesis of Poly[100(DMCD)75(BD or HD)25(HBE)]

One non-triptycene analog (depicted in Scheme 5) with the same composition was synthesized for comparative purposes. The same experimental procedures were applied.

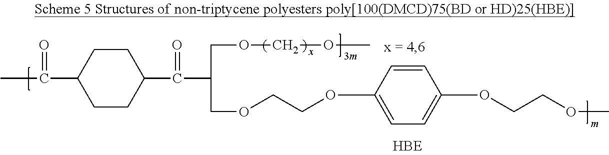

### 2.3.7 Synthesis of Poly[100(DMCD)75(BD)25(BHPS or BHPC or BHPT)]

The other non-triptycene analogs (depicted in Scheme 6) with the same compositions were synthesized for comparative purposes. The same experimental procedures were applied.

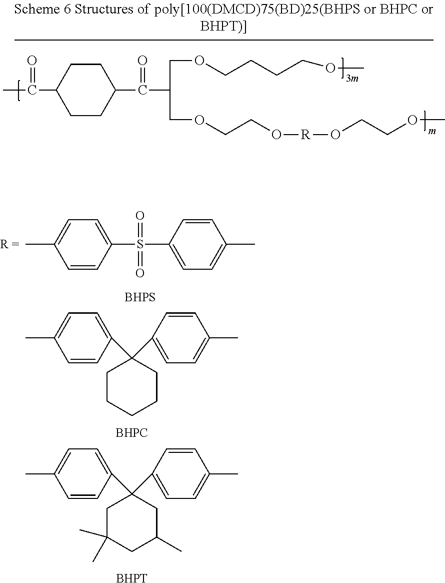

### 3. Results and Discussion

### 3.1 Selection of Monomers

The synthetic route to triptycene-1,4-hydroquione-bis(2-hydroxyethyl) ether (3) is shown in Scheme 1. Anthracene was reacted with quinone across the 9,10 position to yield triptycene-1,4-quinone (1).^{15,16} When treated with HBr in glacial acetic acid, triptycene-1,4-quinone (1) gives triptycene-1,4-hydroquinone (2) with high yield.¹⁵ This bisphenol can be readily and inexpensively converted to the primary alcohol triptycene-1,4-hydroquione-bis(2-hydroxyethyl) ether (3) in high yield by reaction of the phenol OH group with ethylene carbonate.⁹ To the best of our knowledge, (3) has not been reported in the literature and is a new monomer. Triptycene (3) with primary alcohol groups is required for polyesters in diol-diester polycondensations because it is well known that the direct melt polycondensation of bisphenols is a low yield reaction.⁹

Bis[4-(2-hydroxyethoxy)-phenyl]4,4′-cyclohexylidene (BHPC) was obtained in much higher yield than the literature.¹⁸ 1,4-DMCD (a cis/trans ratio of 3 to 1) as the diacid unit was used in this study to improve the solubility of polymers, while maintaining the linear 1,4-enchainment mode.

### 3.2 Copolyester Composition by ¹H NMR Spectroscopic Analysis

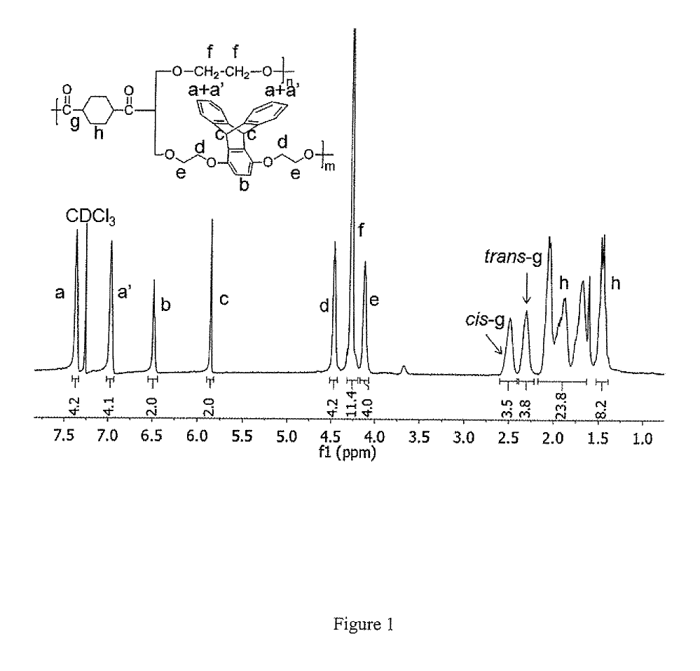

FIG. 1 shows the ¹H NMR spectra of a representative copolyester based on DMCD, EG and TD with a targeted molar ratio of 100:75:25. The diols within the copolyester chains are assumed to react in a random fashion. In brief, peak “a”, “a′”, “b” and “c” are assigned to the protons of the triptycene group. Peaks “d” and “e” are the methylene group adjacent to the oxygen at the TD unit. Peak “f” is assigned to the methylene group adjacent to the oxygen at the EG unit (single peak), and the cis/trans ratio of DMCD was determined by comparing the á-hydrogens on the cis and trans isomers. Some isomerization of DMCD from cis to trans occurred during polymerization (final mole ratio of cis/trans=1/1). These broad peaks “h” come from the protons on carbons in the rings. The peak area ratio of “f” to “e” give 74% EG and 26% TD. The ¹H NMR spectrum of each of these polymers showed good agreement of its actual composition with the targeted composition.

3.3 Thermal Properties Analysis of Copolyesters Poly[100(DMCD)(100−x)(EG)×(TD)]

All polymers became highly viscous when the polymerization proceeded to high conversion, which typically took about 6 h. Some of the copolyesters started to take on a yellow color due to the titanium catalyst. The semi-aromatic copolyester samples were soluble in common chlorinated solvents, such as dichloromethane and chloroform, as expected from the amorphous polyester structure. In order to minimize the effect of physical aging of the respective polyesters on thermal and mechanical properties, all polyester film samples were run as soon as possible after they were made by compression molding. The thermal properties, the molecular weights and tensile properties at ambient temperature (23-25° C.) are summarized in Table 1.

TABLE 1
Characterization results of triptycene and non-triptycene copolyesters at 25° C.

| | | | | | Tensile | Tensile | |
|---|---|---|---|---|---|---|---|
| Polyester | TGA T_{d•} | SEC | | DSC | stress at | strain at | |
| composition | 5% weight | Mₙ | | T_{g} | break | break | Modulus |
| (¹H NMR) | loss (° C.) | (g/mol) | M_{w}/Mₙ | (° C.) | (MPa) | (%) | (MPa) |
| 100(DMCD) | 307 | 51,000 | 2.7 | 15 | | | |
| 100(EG) | | | | | | | |
| 100(DMCD) | 384 | 54,000 | 3.1 | 69 | 48 ± 4 | 8 ± 1 | 1475 ± 137 |
| 74(EG)26(TD) | | | | | | | |
| 100(DMCD) | 384 | 17,000 | 2.7 | 99 | | | |
| 49(EG)51(TD) | | | | | | | |
| 100(DMCD) | 385 | 9,500 | 2.9 | 118 | | | |
| 26(EG)74(TD) | | | | | | | |
| 100(DMCD) | 372 | 43,000 | 3.1 | 23 | 6 ± 0.7 | 1920 ± 76 | 0.9 ± 0.1 |
| 74(EG)26(HBE) | | | | | | | |
| 100(DMCD) | 372 | 33,000 | 2.5 | 27 | | | |
| 49(EG)51(HBE) | | | | | | | |
| 100(DMCD) | 372 | 106,000 | 2.6 | 31 | | | |
| 26(EG)74(HBE) | | | | | | | |
| 100(DMCD) | 375 | 25,500 | 2.2 | 44 | 32 ± 2 | 319 ± 14 | 1169 ± 21 |
| 74(BD)26(TD) | | | | | | | |
| 100(DMCD) | 368 | 20,000 | 1.9 | 4 | | | |
| 74(BD)26(HBE) | | | | | | | |
| 100(DMCD) | 370 | 24,000 | 2.0 | 25 | 11 ± 1 | 494 ± 47 | 50 ± 3 |
| 75(HD)25(TD) | | | | | | | |
| 100(DMCD) | 368 | 27,800 | 2.0 | 47 | 43 ± 3 | 10 ± 2 | 734 ± 27 |
| 65(HD)35(TD) | | | | | | | |
| 100(DMCD) | 360 | 24,300 | 2.5 | 77 | | | |
| 50(HD)50(TD) | | | | | | | |
| 100(DMCD) | 346 | 19,000 | 2.1 | −6 | | | |
| 75(HD)25(HBE) | | | | | | | |
| 100(DMCD) | 331 | 20,100 | 1.9 | 32 | 20 ± 2.0 | 433 ± 41 | 316 ± 30 |
| 74(BD)26(BHPS) | | | | | | | |
| 100(DMCD) | 359 | 20,400 | 2.0 | 26 | 15 ± 0.7 | 688 ± 27 | 4 ± 0.2 |
| 74(BD)26(BHPC) | | | | | | | |
| 100(DMCD) | 357 | 19,500 | 2.0 | 36 | 19 ± 0.7 | 249 ± 18 | 673 ± 46 |
| 75(BD)25(BHPT) | | | | | | | |

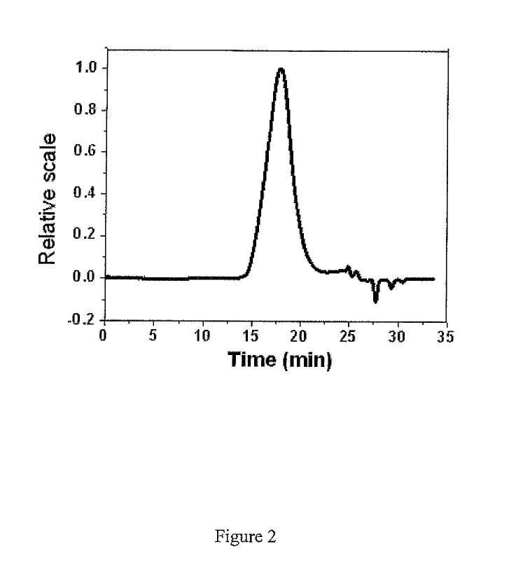

An examination of Table 1 shows that most of the copolyesters displayed high molecular weights as well as PDIs of 2.5-3.1, which are typical for melt polymerization polyesters. The SEC trace of poly[100(DMCD)74(EG)26(TD)] by the refractive index detector is shown in FIG. 2 as representative of these samples. The presence of small peaks, following the main sharp peak, suggests the presence of some cyclic oligomers in the polyester. Molecular weights of polyesters, containing DMCD, EG, and TD, decrease with decreasing EG content. However, T_{g} increases with increasing TD content. As shown in Table 1, the 5% weight loss (T_{d}) for all TD-containing polymers was higher than those of the corresponding non-TD analogs, as expected from the more highly aromatic structure of TD. For example, poly[100(DMCD)74(EG)26(TD)] showed a T_{d} at 384° C., whereas poly[100(DMCD)74(EG)26(HBE)] displayed a T_{d} of 372° C. These data indicate that copolyesters containing TD have marginally higher thermal stability than non-triptycene analogs. When the triptycene unit was incorporated into the polyester, the T_{g}s were remarkably increased when compared to those of the non-triptycene polyesters. From Table 1, the incorporation of 26 mol % HBE into the DMCD/EG backbone only raised the T_{g} 8° C., whereas TD at the same incorporation level raised the T_{g} 54° C. This is consistent with the bulky structure of TD. The absence of a melting peak in all DSC traces is also indicative of the amorphous character of the copolyesters. It is difficult to obtain high molecular weight copolyesters with an incorporation level of 70 mol % or more TD due to the non-volatility of TD.

### 3.4 Mechanical Property Analysis of TD Copolyesters

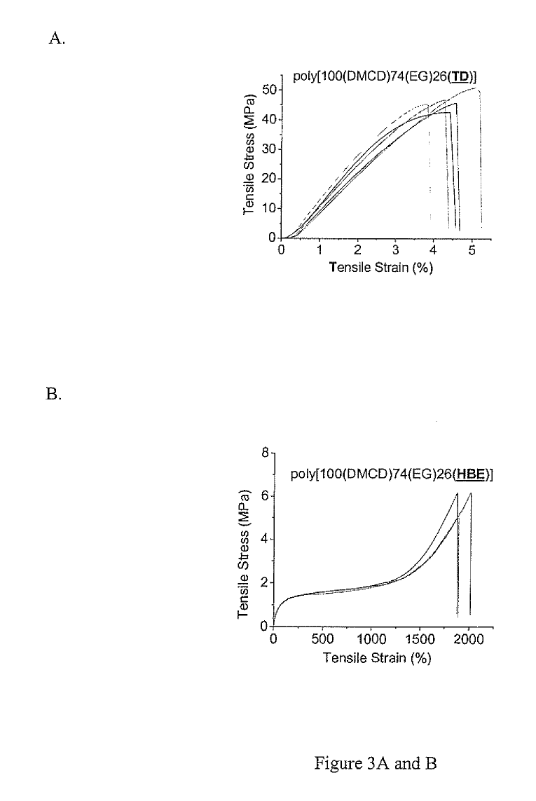

Tensile data at ambient temperature revealed that poly[100(DMCD)74(EG)26(TD)] has a higher modulus and yield stress than poly[100(DMCD)74(EG)26(HBE)], which is a flexible material (FIG. 3 and Table 1). But the low elongation to break (14%) of poly[100(DMCD)74(EG)26(TD)] indicated that this triptycene polyester is brittle under these same conditions. Therefore, we replaced EG by BD and kept the TD composition the same. A similar polymer was also prepared with hexanediol so that the effects of aliphatic spacers could be elucidated. We observed significantly different properties with the longer straight chain diols BD and HD.¹¹ These characterization results are also summarized in Table 1.

Table 1 demonstrated that, as expected, the T_{g} of triptycene copolyesters decreases when the carbon number of the linear aliphatic co-diol increases. Average molecular weights also decreased with the longer chain aliphatic diols because of their lower volatility than EG, making it more difficult to drive the conversion. The TD-containing polymers poly[100(DMCD)75(BD or HD)25(TD)] exhibited higher thermal stabilities than the corresponding non-triptycene analogs poly[100(DMCD)75(BD or HD)25(HBE)]. Tensile data in Table 1 revealed that poly[100(DMCD)74(BD)26(TD)] has a higher modulus and stress to yield than poly[100(DMCD)75(HD)25(TD)], which has more elastic-like properties. High elongation to break (319%) and high modulus (1.17 GPa) of poly[100(DMCD)74(BD)26(TD)] indicated that it is ductile and a relatively tough material. A decrease of HD content in triptycene copolyester from 75 mol % to 65 mol % results in a brittle material with low elongation to break (10%) at ambient temperature. When the HD content is lowered to 50 mol %, poor films are obtained and tensile data are not available due to the fragility of this triptycene polyester sample.

### 3.5 Mechanical Property Analysis of Copolyesters Containing BHPS, BHPC or BHPT as Comparative Examples

Numerous bisphenol derivatives have been synthesized and incorporated into polymer backbones to increase the T_{g} for high performance materials.^{9,19} BHPS is commercially available; BHPC and BHPT were synthesized as described in the synthesis section. In this study, they were incorporated into identical polyester backbones by replacing the triptycene units with the respective bisphenol derivatives, BHPS, BHPC or BHPT. The properties of their copolyesters are shown in Table 1.

From Table 1, we can see that poly[100(DMCD)74(BD)26(TD)] still has the highest thermal stability and highest modulus among these copolyesters. These data thus confirmed that the incorporation of TD into the polyester backbone can increase T_{g} due to its rigid structure. The glassy solid BHPT possesses an amorphous and bulky structure due to the three pendent methyl groups. When the BHPT concentration was also fixed at 25 mol %, the modulus of the corresponding copolyester was significantly less than the 25 mol % TD copolyester. From the tensile test data of poly[100(DMCD)74(BD)26(BHPC)], we conclude that it has some elastic-like properties with a low modulus (only 4 MPa) since T_{g} is very close to the ambient temperature, at the temperature where the tensile measurements were run. The copolyester based on BHPS, poly[100(DMCD)74(BD)26(BHPS)] has a lower T_{g} and modulus and a higher elongation than the TD copolyester.

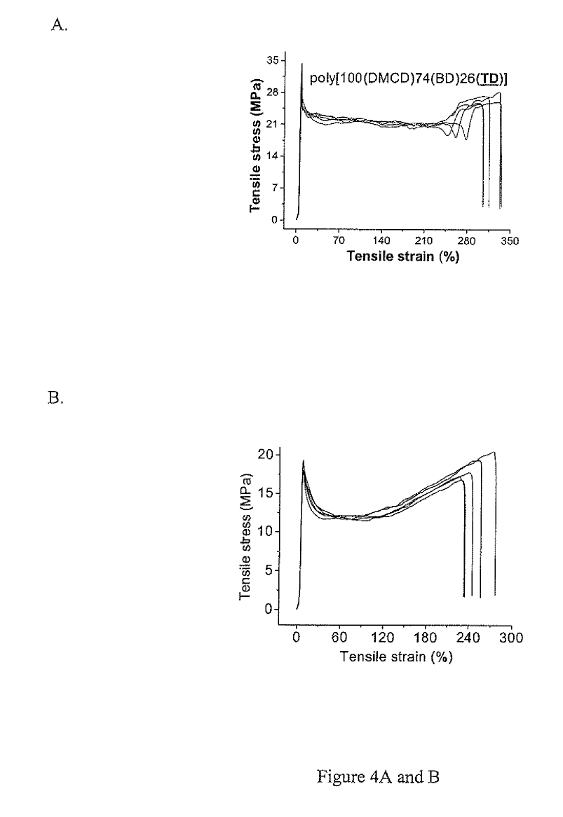

From the above tensile curves for poly[100(DMCD)74(BD)26(BHPT)] (FIG. 4), the copolyester based on BHPT was found to be a high modulus and ductile material with a yield point. However, when compared to poly[100(DMCD)74(BD)26(BHPT)], the triptycene copolyester poly[100(DMCD)74(BD)26(TD)] exhibits an improvement in both the elongation to break and modulus which are important and useful properties application of these copolyesters containing TD.

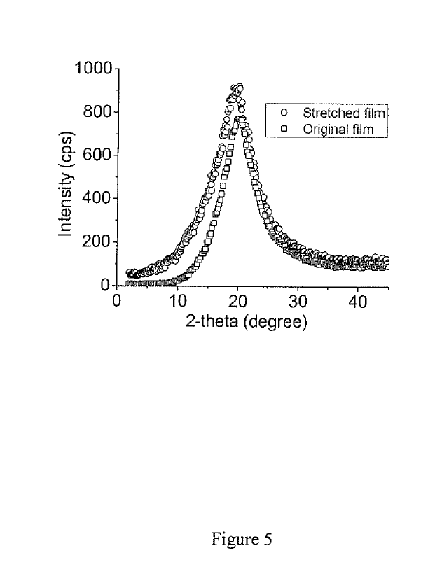

The tensile curve of poly (100(DMCD)74(BD)26(TD) shown in FIG. 4 demonstrated considerable strain hardening at high elongations. In order to verify if any crystallization is induced during the deformation, we obtained the x-ray diffraction of the elongated polymer film, which was maintained in the stretched condition during the measurement. FIG. 5 shows the x-ray diffraction traces for both the original and stretched samples. No significant difference between these films is observed. The broad diffuse peaks (almost across 20°) indicate that both samples are amorphous.

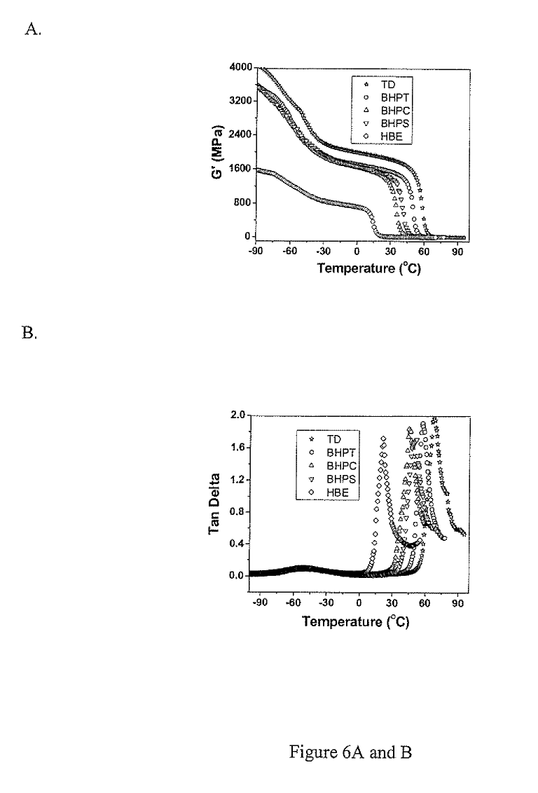

DMA spectra of triptycene and various non-triptycene polyesters are presented in FIG. 6. The plots of storage modulus versus temperature indicate that all polyesters possess an expected glassy storage modulus (above 1 GPa) except for HBE-containing polyester. The alpha peaks in the tan delta curves, accompanied by a sharp decrease in modulus, correspond to the T_{g}'s of the respective polyesters. The triptycene polyester displays the highest glassy storage modulus (above 1.8 GPa) and T_{g} (66° C. from tan delta), together with the widest to glassy plateau well past room temperature among these polyesters, while the HBE-containing polyester shows the lowest glassy DMA modulus of 0.7 GPa and T_{g} of 21° C. The tan delta T_{g} was about 20° C. higher than the DSC T_{g} as expected. All copolyesters exhibit the existence of a secondary relaxation tan a peak from −57 to −51° C. with about the same intensity. The conformational changes of the cyclohexyl units in the polyester backbones are the origin of sub-T_{g} motions. It is of interest to note that other cyclohexyl group-containing polyesters, such as the poly(1,4-cyclohexylenedimethylene terephthalate) (PCT), were shown by Yee et al. to have a weak transition in this region as well.²⁰

### 4. Conclusions

In summary, we have described the synthesis of triptycene-containing polyesters using a novel primary triptycene diol and we have also characterized properties of these new materials. All TD-containing polymers in this study have higher thermal stability by TGA and higher T_{g}'s than the corresponding non-triptycene analogs. The T_{g} of TD-containing polymers increased with increasing TD content. The results from tensile tests revealed that poly[100(DMCD)74(EG)26(TD)] is rigid but brittle at ambient temperature. However, the copolyester poly[100(DMCD)74(BD)26(TD)] was found to simultaneously possess high modulus and excellent ductility at ambient temperature. It indicates that its short flexible spacer (butane unit) in combination with triptycene units can also promote an improved ambient temperature modulus and enhanced ductility. These results are not predicted by the earlier reference (11) where a long chain diol (decane diol) was incorporated into the polyester backbone.

### REFERENCES FOR EXAMPLE 1

(1) Park, K. H.; Tani, T.; Kakimoto, M. A.; Imai, Y. J. Polym. Sci. Part A.: Polym. Chem. 1998, 36, 1767.
(2) Liaw, D. J.; Liaw, B. Y.; Yang, C. M. Macromolecules 1999, 32, 7248.
(3) Liaw, D. J.; Hsu, P. N.; Chen, W. H.; Lin, S. L. Macromolecules 2002, 35, 4669.
(4) Chern, Y. T.; Wang, W. L. Macromolecules 1995, 28, 5554.
(5) Chern, Y. T.; Shiue., H. C.; Kao, S. C. J. Polym. Sci. Part A.: Polym. Chem. 1998, 36, 785.
(6) Van Reenen, A. J.; Mathias, L. J.; Coetzee, L. Polymer 2004, 45, 799.
(7) Hsiao, S. H.; Li, C. T. J. Polym. Sci. Part A.: Polym. Chem. 1999, 37, 1435.
(8) Weyland, H. G.; Hoefs, C. A. M.; Yntema, K.; Mijs, W. J. Eur. Polym. J. 1970, 6, 1339.
(9) Turner, S. R.; King, B.; Ponasik, J.; Adams, V.; Connell, G. High Perform. Polym. 2005, 17, 361.
(10) Keohan, F. L.; Freelin, R. G.; Riffle, J. S.; Yilgor, I.; McGrath, J. E. J. Polym. Sci.: Polym. Chem. Ed. 1984, 22, 679.
(11) Tsui, N. T.; Paraskos, A. J.; Torun, L.; Swager, T. M.; Thomas, E. L. Macromolecules 2006, 39, 3350.
(12) Tsui, N. T.; Yang, Y.; Swager, T. M.; Thomas, E. L. Polymer 2008, 49, 4703.
(13) Klanderman, B. H.; Faber, J. W. H. J. Polym. Sci.: Part A-1 1968, 6, 2955.
(14) Hoffmeister, E. K.; Kropp, J. E.; McDowell, T. L.; Michel, R. H.; Rippie, W. L. J. Polym. Sci. Part A-1 1969, 7, 55.
(15) Bartlett, P. D.; Ryan, M. J.; Cohen, S. G. J. Am. Chem. Soc. 1942, 64, 2649.
(16) Adams, H.; Jones, S.; Jimenez, O. I. Org. Biomol. Chem. 2006, 4, 2296.
(17) Wilcox, C. F.; Roberts, F. D. J. Org. Chem. 1965, 30, 1959.
(18) Hait, S. B.; Sivaram, S. Polym. Int. 1998, 47, 439.
(19) Schmidhauser, J.; Sybert, P. D. In Handbook of Polycarbonate Science and Technology; Marcel Dekker, Inc.: Plastics Engineering, New York, 2000; Vol. 56, p 61.
(20) Chen, L. P.; Yee, A. F.; Goetz, J. M.; Schaefer, J. Macromolecules 1998, 31, 5371.

### EXAMPLE 2

### Synthesis and Properties of Triptycene-Containing Polyurethanes

### 1. Introduction

The primary triptycene diol provides a new monomer for incorporating into polyesters and polyurethanes. We initially studied the incorporation of this new monomer into polyesters by melt phase polymerizations and observed significantly higher Tgs and modulus values while maintaining excellent elongation properties (see Example 1). In this Example we describe research on the incorporation of TD into polyurethanes.

### 2. Experimental

### 2.1 Materials.

Poly(tetramethylene glycol) (PTMG) oligomer (Terathane, DuPont) with a Mn of 1000 g/mol, 4-butanediol (BDO), hydroquinone bis(2-hydroxyethyl) ether (HQEE, 98%) and 4,4′-methylenebis(phenyl isocyanate) (MDI, 98%) were purchased from Aldrich and dried under vacuum overnight at 60° C. 1,4-Triptycene hydroquinone was from ICx Technologies, Inc. TD was prepared as described in Example 1. Anhydrous tetrahydrofuran (THF, Aldrich, ≧99.9%) and anhydrous n-butanol (Aldrich, 99.8%) were used as received.

### 2.2 Synthesis of Soft-Segment Oligomeric Diols Containing Triptycene and Comparative Bisphenols

Synthesis of mesylate end-capped PEG400 copolymers with phenyl, 4,4′-biphenol, triptycene structural units. The synthetic scheme of the series of copolymers is shown in FIG. 2. [1] The procedure is similar with the synthesis of triptycene-containing PEG copolymer. A typical synthetic procedure is shown below using triptycene hydroquinone (TH) as an example. The molar ratio of PEG400-dimesylate to TH is 2:1. A 100 mL two-necked round bottom flask was charged with PEG-dimesylates (1.4 mmol), TH (0.7 mmol), anhydrous potassium carbonate (3.5 mmol) and anhydrous dimethylformamide (DMF) (6 ml). The reaction mixture was stirred at 80° C. under argon for 24 h. The reaction was cooled to room temperature and then decanted into water. Then the mixture was extracted by dichloromethane (DCM). The remaining DMF solvent was washed by water for three times. DCM was evaporated on a rotary evaporator and yielded yellowish viscous oil. The crude product was redissolved by chloroform and precipitated in methanol under dry ice bath (acetone). The final product was dried overnight in vacuum oven at 60° C. The structure of final product was characterized by 1H NMR. The integration of the 1H NMR spectrum suggested that the product is a mixture of copolymers with different number of repeating units,

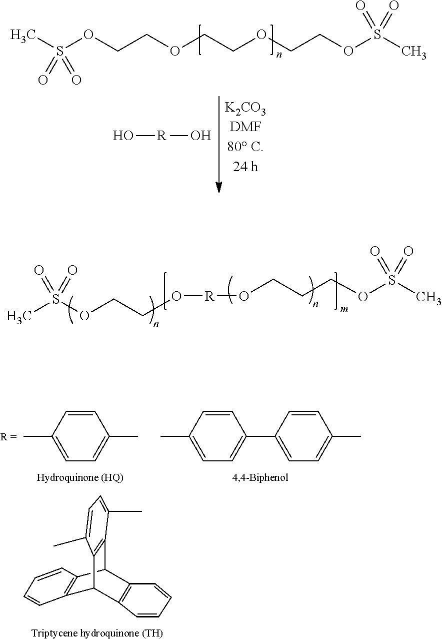

2.3 Hydrolysis of Mesylate End-Capped PEG400 Copolymers with Phenyl, 4,4′-Biphenol, Triptycene Structural Units.

The synthetic scheme of the series of copolymers is shown in FIG. 1 which follows a published literature procedure [2]. A typical procedure is shown below using triptycene hydroquinone (TH) as an example, hydrolysis of PEG copolymer dimesylate containing phenyl groups was using the same method. A mixture of PEG copolymer dimesylate containing triptycene units (1.2 g, 2.2 mmol), K2CO3 (1.04 g, 7.5 mmol), water (0.55 ml) and DMF (5.4 ml) were charged into 100 ml two-necked round bottle flask. The reaction mixture was stirred at 125° C. under argon for 12 h. The reaction was cooled to room temperature and then decanted into water. Then the mixture was extracted by dichloromethane (DCM). The organic layer was washed by brine (3×50 ml) and water (3×50 ml) to remove remaining DMF, and then dried by magnesium sulfate. Dark brown viscous oil was obtained by evaporating DCM and further vacuum dried at 60° C. for 24 h. The structure of final product was characterized by 1H NMR. The integration of the 1H NMR spectrum suggested that the product is a mixture of copolymers with different number of repeating units, which is also confirmed by SEC analysis.

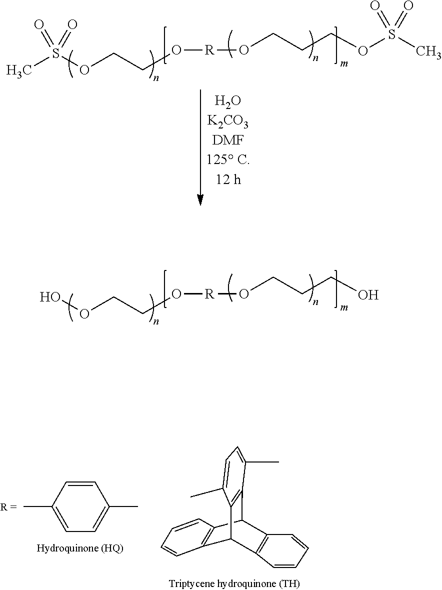

### 2.4 Synthesis of Polyurethanes

Polyurethanes were prepared by the prepolymer method. In the first step, PTMG and MDI were charged in three-necked round bottom flask under argon protection. The flask was heated to 80° C. with stirring until all solids had melted to give a clear liquid. Stirring was continued for 2.5 h. In the second step, chain extenders (BDO, HQEE, TD) were dissolved in anhydrous THF and added into the reaction system. The chain extension proceeded at 80° C. for 2.5 h. Polyurethane films were obtained by casting the clear, homogeneous solution in a Teflon mold at 60° C. for 1 day, followed by 1 day under vacuum at 60° C. Films were cut into dog-bone shape species roughly 20 mm in length and 4 mm in width for testing.

### 3. Results and Discussion

We have prepared a series of polyurethanes based on the prepolymer of PTMG, soft segment diol oligomers containing triptycene and MDI, HDI and other disocyanates. These prepolymers were chain extended by 1,4-butanediol, HQEE, TD, the new TD containing soft segments and the thermal and mechanical properties were evaluated.

The thermal properties of polyurethanes based on different chain extenders were investigated by thermogravimetric analysis (TGA) and differential scanning calorimetry (DSC). The results in Table 3 revealed the impact of introducing triptycene diol into the polyurethane structure. According to the TGA data, no dramatic differences were observed for the three polyurethanes.

TABLE 3
Thermal Properties of PU samples

| | T_{d} 5% | T_{g} (soft | T_{g} (hard |
|---|---|---|---|
| Polymers | weight loss (° C.) | segment (° C.) | segment (° C.) |
| PU-BDO | 300 | −42 | 99 |
| PU-HQEE | 304 | −47 | 93 |
| PU-TD | 300 | −65 | 87 |

No crystallization peak was observed in any of the samples when tested by DSC, likely due to the relatively short length of the soft segment. The introduction of TD appeared to interrupt the formation of hydrogen bonds in the hard segment as shown by the reduction in T_{g}. Since hydrogen bonds would restrict the mobility of polymer chains, with few hydrogen bonds, triptycene-containing PU showed lower T_{g} for both of soft and hard segments. The reduction in hydrogen bonding could also be observed in the Fourier transform infrared (FTIR) spectra of the TD based PUs vs. the two controls (not shown).

Tensile measurements were employed to investigate the mechanical properties of polyurethane samples. The results, in Table 4, suggest that the Young's modulus was enhanced in TD containing polymers whereas elongation values appeared to decrease in comparison to the controls. The introduction of TD into the hard segment led to enhanced modulus due to its bulky structure.

TABLE 4
Tensile Properties for PU with Different Chain Extenders

| | Tensile stress | Tensile strain | Young's Modulus |
|---|---|---|---|
| Polymers | at break (MPa) | at break (%) | (MPa) |
| PU-BDO | 33.8 ± 4.3 | 1296 ± 175 | 3.8 ± 0.4 |
| PU-HQEE | 69.0 ± 4.0 | 1039 ± 66 | 14 ± 1.5 |
| PU-TD | 51.3 ± 6.5 | 721 ± 31 | 20.4 ± 2.5 |

The combination of high ductility with enhanced modulus when TD is placed in the PU hard segment is an excellent combination of properties for applications as matrix polymers for fiber composites.

### REFERENCES FOR EXAMPLE 2

1. Hong, K.-C.; Kim, J.; Bae, J.-Y. Polymer Bulletin 2000, 44, 115.
2. Pejanovi\grave{c}, V.; Piperski, V.; Uglje\check{s}ić-Kilibarda, D.; Tasić, J.; Da\check{c}ević, M.; Medić-Mija\check{c}ević, L.; Gunić, E.; Popsavin, M.; Popsavin, V. European Journal of Medicinal Chemistry 2006, 41, 503.

All patents, patent application and published articles cited herein are hereby incorporated by reference in entirety.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. Triptycene-1,4-hydroquinone-bis(2-hydroxyethyl) ether (TD).

2. A polyester comprising triptycene-1,4-hydroquinone-bis(2-hydroxyethyl) ether (TD).

3. The polyester of claim 2, wherein said polyester is a polyesteramide.

4. A method of making a polyester, comprising
reacting triptycene-1,4-hydroquinone-bis(2-hydroxyethyl) ether (TD) with at least one dicarboxlic acid under conditions which permit copolymerization of said TD and said at least one dicarboxlic acid via the formation of ester bonds.

5. The method of claim 4, wherein said step of reacting is carried out via melt phase polycondensation.

6. The method of claim 4, wherein said step of reacting is carried out using at least one additional diol that is not TD.

7. A method of making a polyester, comprising
reacting triptycene-1,4-hydroquinone-bis(2-hydroxyethyl) ether (TD) with at least one diester of a dicarboxylic acid.

8. A polyurethane comprising triptycene-1,4-hydroquinone-bis(2-hydroxyethyl) ether (TD).

9. The polyurethane or claim 8, wherein said TD is present in a soft segment of said polyurethane.

10. A method of making a polyurethane, comprising
reacting at least one polyisocyanate and at least one polyol in the presence of triptycene-1,4-hydroquinone-bis(2-hydroxyethyl) ether (TD) as a chain extender, under conditions which permit polymerization of said at least one polyisocyanate and said at least one polyol via the formation of urethane bonds.

11. The method of claim 10 wherein the at least one polyol contains one or more TD units in the backbone.
